(19) United States
(12) Patent Application Publication
(10) Pub. No.: US 2020/0203854 A1
(43) Pub. Date: Jun. 25, 2020

(54) CABLE ASSEMBLY, CABLE HOLDER, AND PRODUCTION METHOD FOR CABLE ASSEMBLY

(71) Applicant: Molex, LLC, Lisle, IL (US)

(72) Inventor: Naoyoshi TAMURA, Yamato (JP)

(73) Assignee: Molex, LLC, Lisle, IL (US)

(21) Appl. No.: 16/711,446

(22) Filed: Dec. 12, 2019

(30) Foreign Application Priority Data
Dec. 20, 2018 (JP) 2018-238327

(51) Int. Cl. H01R 4/02; H01R 4/70; H01R 43/02; H05K 7/02

(52) U.S. Cl. CPC H01R 4/023; H01R 4/70; G01J 1/0219; H05K 7/02; H01R 43/02

(57) ABSTRACT

A cable assembly is provided which includes a sensor body, a sensor having terminals disposed on a terminal array surface of the sensor body, and a cable holder fixed to the terminal array surface of the sensor body. The cable holder has protruding connection grooves provided at positions corresponding to the terminals of the cable holder and extending from a connection surface on the sensor side toward a cable extension surface on the opposite side. The cable assembly further includes a cable which is joined to the cable holder by soldering inner core wires to the terminals and the connection grooves.

### RELATED APPLICATIONS

This application claims priority to Japanese Application No, 2018-238327, filed on Dec. 20, 2018, which application is incorporated herein by reference in its entirety.

### TECHNICAL FIELD

The present disclosure relates to a cable assembly including an electronic device such as a sensor, a cable connected to the electronic device, and a cable holder for supporting the electronic device and the cable; a cable holder which is fixed to an electronic device in order to connect a cable to the electronic device and supports the cable; and a production method for a cable assembly for connecting a cable to an electronic device using a cable holder.

### BACKGROUND ART

Conventionally, when connecting a signal or power cable to a sensor such as an individual imaging element via a circuit board, there is a known tip part of an electronic endoscope which enables a connection operation to be performed while the length of a solder margin is stably secured, and which enables a signal line connection to be soldered to the circuit board with sufficient strength for example, see Patent Document 1.

Specifically, Patent Document 1 discloses a tip part of an electronic endoscope in which the tip portion of a signal line drawn in a direction facing a substrate surface of a circuit board from a tip of a signal cable inserted into an insertion part is connected to a connection terminal part provided on an outer edge part of the circuit board, wherein the connection terminal part of the circuit board is formed with a shape that is a groove shape into which the tip portion of the signal line is fitted, the shape having an abutting surface at which the tip surface of the signal line abuts.

In the configuration of Patent Document 1, a signal line of the signal cable is soldered to the circuit board, and the signal cable and an individual imaging element are electrically connected via the circuit board. Electronic components are disposed on the circuit board, and the circuit board and the individual imaging element are further connected by leads so as to electrically connect the signal cable and the individual imaging element.

Such a configuration is relatively large, as the electronic components are disposed on the circuit board, however when the individual imaging element is a very small electronic device such as a chip-like CMOS (Complementary Metal-Oxide Semiconductor) image sensor, the circuit board must be made small in accordance with the sensor or the like. This leads to a problem in that it is difficult to connect the signal cable to a sensor or the like with a similar means.

Patent Document 1: Japanese Unexamined Patent Application Publication No. 2006-68057

### SUMMARY

Therefore, the present disclosure was conceived in light of the problems described above, and an object thereof is to provide a cable assembly, a cable holder, and a production method for a cable assembly which, even when attempting to connect a signal or power cable to a small electronic device such as a chip-type sensor, for example, can facilitate the connection of the cable to the electronic device and secure sufficient strength to fix the electronic device and the cable.

The present disclosure was proposed to achieve the object described above, and one mode of the present disclosure is a cable assembly including an electronic device, a cable connected to the electronic device, and a cable holder for supporting the electronic device and the cable, Herein, the electronic device includes a terminal array surface on which terminals to be electrically connected to the cable are arranged; the cable holder includes a connection surface facing the terminal array surface, and a side surface having a connection groove formed with an opening corresponding to the array positions of the terminals on the connection surface so as to support the cable in a direction intersecting the terminal array surface, and the connection groove is subjected to surface treatment to allow an electrical connection with a core wire of the cable; the terminals are provided with a convex conductive body housed in the connection groove; an adhesive layer is provided between the terminal array surface and the connection surface; and the cable holder is translucent.

Another mode of the present disclosure is a cable holder including: a connection surface which is a plane facing a terminal array surface on which terminals of an electronic device to which a cable is to be connected are arranged; a cable extension surface from which the signal cable extends; and a side surface connecting the connection surface and each side of the cable extension surface. Herein, the side surface includes a connection groove formed continuously so as to open toward the side surface side by forming an opening in the connection surface and the cable extension surface; and the holder is translucent.

Yet another mode of the present disclosure is a production method for a cable assembly. This method includes: arranging a plurality of terminal array surfaces on which terminals of an electronic device are arranged so as to face upward; applying a photocurable adhesive to locations other than the terminals of the terminal array surface lower than convex conductive bodies formed on the terminals; mounting a translucent cable holder so as to spread the applied photocurable adhesive to the back surface; adhesively fixing a sensor and the cable holder by irradiating light from an upper surface on the cable holder side; installing a core wire of the cable in a connection groove formed in the cable holder; and connecting the core wire, the connection groove, and the terminals.

With the present disclosure, it is possible to provide a cable assembly, a cable holder, and a production method for a cable assembly which, even in the case of a small electronic device such as a chip-type sensor, for example, can facilitate the connection of a cable to the electronic device and secure sufficient strength to fix the electronic device and the cable.

### BRIEF DESCRIPTION OF DRAWINGS

FIG. 1 is an exploded perspective view of a cable assembly of a first embodiment.

FIG. 2 is a perspective view illustrating a state after the respective elements in FIG. 1 are connected to one another.

FIG. 3 is a cross-sectional view along line B-B in FIG. 2.

FIGS. 4A and 4B are perspective views illustrating a sensor extracted from FIG. 1, wherein FIG. 4A illustrates a state in which solder balls are applied to the sensor, and FIG. 4B illustrates a state in which a UV curing adhesive is further applied.

FIG. 5 is a perspective view illustrating a state before a cable holder is mounted on the sensor in FIG. 4B.

FIG. 6 is a perspective view illustrating a state after the cable holder is mounted on the sensor in FIG. 4B.

FIGS. 7A and 7B are drawings illustrating the configuration of the sensor of FIG. 4B, wherein FIG. 7A is a plan view and FIG. 7B is a cross-sectional view along line C-C in FIG. 7A.

FIGS. 8A and 8B are drawings illustrating the configuration of the sensor and the cable holder in FIG. 6, wherein FIG. 8A is a plan view and FIG. 8B is a cross-sectional view along line D-D in FIG. 8A.

FIGS. 9A and 9B are drawings illustrating the configuration of the sensor and the cable holder in FIG. 6, wherein FIG. 9A is a cross-sectional view along line H-H in FIG. 9B, and FIG. 9B is a side view.

FIG. 10 is a perspective view illustrating a cable assembly of a second embodiment in a state before the cable assembly is mounted to a sensor.

FIGS. 11A and 11B are drawings illustrating the configuration of a state after the cable holder is mounted to the sensor in FIG. 10, wherein FIG. 11A is a plan view and FIG. 11B is a cross-sectional view along line E-E in FIG. 11A.

FIGS. 12A and 12B are drawings illustrating the configuration of a state after the cable holder is mounted to the sensor in FIG. 10, wherein FIG. 12A is a cross-sectional view along line J-J in FIG. 12B, and FIG. 12B is a side view.

FIGS. 13A and 13B are drawings illustrating a sensor and a cable holder according to a cable assembly of a third embodiment, wherein FIG. 13A is a perspective view illustrating the sensor, and FIG. 13B is a perspective view illustrating a state after the cable holder is mounted to the sensor in FIG. 13A.

FIGS. 14A and 14B are drawings illustrating the configuration of the sensor and the cable holder in FIG. 13B, wherein FIG. 14A is a plan view and FIG. 14B is a cross-sectional view along line F-F in FIG. 14A.

### DETAILED DESCRIPTION OF THE PREFERRED EMBODIMENTS

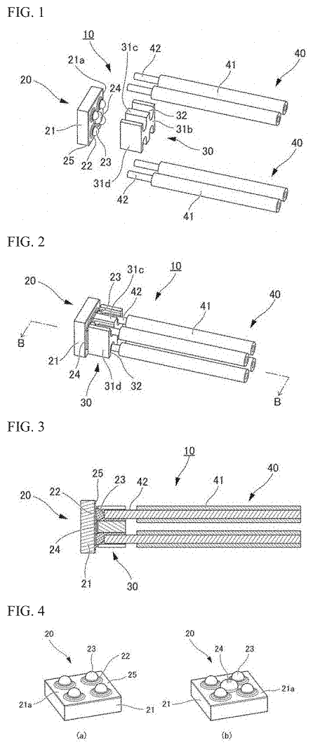

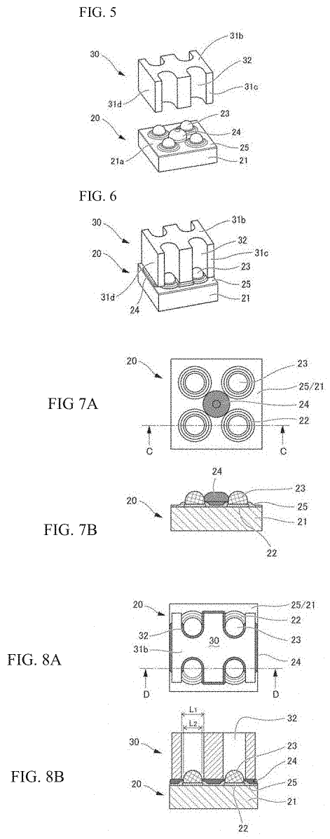

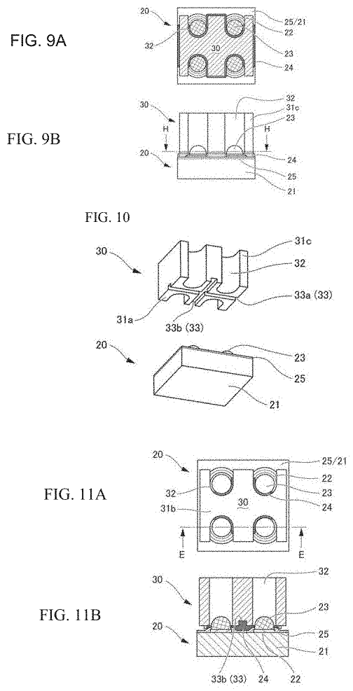

Embodiments of the present disclosure will be described in detail hereinafter with reference to the drawings. FIGS. 1 to 9B illustrate a first embodiment; FIGS. 10 to 12B illustrate a second embodiment; and FIGS. 13A to 14B illustrate a third embodiment. Note that throughout the entire descriptions of the embodiments, the same elements will be described with the same symbols.

Note that expressions indicating directions such as up, down, left, right, front, and back used to described the embodiments are not absolute but rather are relative directions, and although the expressions are appropriate when each part is in the position illustrated in the drawings, the directions should be interpreted differently in accordance with any change in position when the position changes.

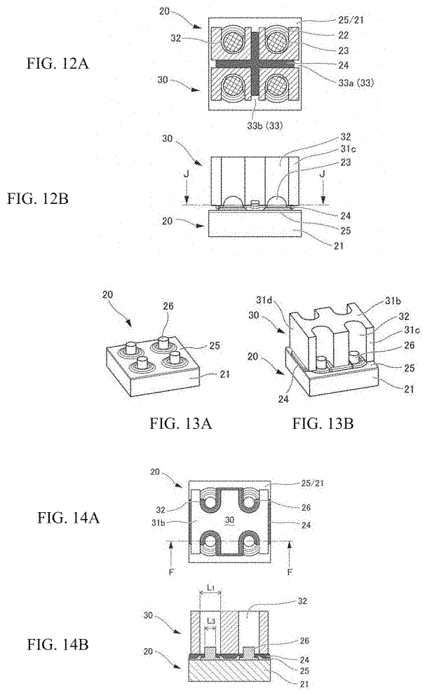

First, the overall configuration of a cable assembly 10 of a first embodiment will be described with reference to FIGS. 1 to 3. FIG. 1 illustrates a state before each element of the cable assembly 10 is connected; FIG. 2 illustrates a state after the respective elements of FIG. 1 are connected to one another; and FIG. 3 illustrates a cross section along line B-B in FIG. 2. As illustrated in FIG. 1, the cable assembly 10 includes a sensor 20, a cable 40, and a cable holder 30 serving as a support for connecting the sensor 20 and the cable 40. Hereinafter, a mode in which four cables 40 are connected to one sensor 20 and a cable holder 30 will be described, however, the present invention is not limited to this mode, as described below.

As an example of an electronic device in the present application, the sensor 20 is, for example, a thin, rectangular plate-shaped chip-type sensor, and four terminals 22 (a conduction pattern, PAD, or the like formed by plating or the like) to which core wires 42 of cables 40 described below are connected are formed on a surface serving as a cable connection side of a sensor body 21 (right side in FIG. 1). This surface is used as a terminal array surface 21a. The periphery of each terminal 22 is covered by a resist 25 (solder resist). Solder balls 23 are applied to the circular surfaces of the terminals 22 not covered by the resist 25 (right side in FIG. 1), and a UV (ultraviolet) curing adhesive 24 is applied to the center portion surrounded by the four solder balls 23.

The cable holder 30 is formed in a rectangular parallelepiped shape including a rectangular connection surface 31a facing the terminal array surface 21a of the sensor, a cable extension surface 31b which is parallel with and of the same shape as the connection surface 31a, a pair of first side surfaces 31c connecting the parallel sides of the connection surface 31a and the cable extension surface 31b, and a pair of second side surfaces 31d connecting the other parallel sides.

The pair of first side surfaces 31c has penetrating connection grooves 32 extending from the connection surface 31a toward the cable extension surface 31b on the opposite side (cable 40 side). The connection grooves 32 are provided at positions corresponding to the array of the terminals 22, and open at the connection surface 31a in alignment with the positions of the terminals 22 of the sensor 20. In this embodiment, the drawings illustrate a mode in which two of each are provided on the pair of first side surfaces 31c on the upper and lower sides.

The connection grooves 32 are formed in a groove shape so that the bottom surface side thereof is opened to the first side surface 31c side with a cylindrical wall surface of a prescribed diameter (L1 in FIG. 8b). The opening shape of the connection grooves 32 at the connection surface 31a is formed larger than the diameter of the solder balls (L2 in FIG. 8b), allowing the solder balls 23 to be easily housed in the connection grooves 32 when the cable holder 30 is mounted on the terminal array surface 21a of the sensor body 21 provided with the solder balls 23.

In this embodiment, the surfaces of the terminals 22 not covered by the resist 25 and the solder balls 23 are formed with the same diameter L2.

As described below, the cable holder 30 is preferably formed from a translucent, UV-permeable material (for example, a material that transmits light such as UV-permeable glass). In this case, since the cable holder 30 is formed from an insulating body, the surfaces of the connection grooves 32 are plated with gold or the like to ensure conductivity and solderability. Each connection groove 32 is electrically independent from the other.

The cable 40 has a cable sheath 41 and a core wire 42 for powering the inside thereof. The sensor 20 side—that is, the end side—of the core wire 42 is exposed so as to be soldered and joined to the terminals 22 of the sensor 20 and the connection grooves 32 of the cable holder 30.

FIGS. 2 and 3 illustrate a state after the cable holder 30 has been adhered and fixed to the sensor 20 described above and the cable 40 is connected. The connection surface 31a of the cable holder 30 is fixed to the terminal array surface 21a of the sensor body 21 by a UV curing adhesive 24. When the cable holder 30 is mounted on the terminal array surface 21a of the sensor 20, the UV curing adhesive 24 spreads between the connection surface 31a of the cable holder 30 and the terminal array surface 21a of the sensor 20, excluding the regions of the terminals 22 where the solder balls 23 are soldered, thereby forming an adhesive layer. As described above, the cable holder 30 is formed from a UV-permeable material, and the cable holder 30 is adhered and fixed to the sensor by the UV curing adhesive 24 when irradiated with UV from the cable holder 30 side.

By adhering using the UV curing adhesive 24, the fixing of the cable holder 30 and the sensor 20 can be achieved with UV irradiation without heating, which is effective to minimize the effects of heat due to soldering or the like on the sensor 20.

The core wire 42 of the cable 40 is mounted in the connection groove 32 in a state in which the tip abuts the solder ball 23 inside the connection groove 32 of the cable holder 30, and is joined to the terminal 22 of the sensor 20 and the plating of the connection groove 32 of the cable holder 30 by soldering so that the sensor 20 and the cable 40 are electrically conductive with one another.

In this way, by assembling the sensor 20 and the cable 40 via the cable holder 30 in which the connection grooves 32 are formed, the connection of the cable in a direction intersecting the terminal array surface 21a of the sensor 20—for example, a direction orthogonal to the terminal array surface 21a—can be achieved easily, and by soldering to the connection grooves 32 of the cable holder 30, the strength with which the sensor 20 and the cable 40 are fixed can be sufficiently ensured.

Next, the details of the sensor 20 and the cable holder 30 will be described with reference to FIGS. 4A to 9B. FIG. 4A illustrates a state in which the solder balls 23 are applied to the terminals 22 of the sensor 20, and FIG. 4B illustrates a state in which a UV curing adhesive 24 is further applied. FIG. 5 illustrates a state before the cable holder 30 is mounted to the sensor 20, and FIG. 6 illustrates a state after the cable holder 30 is mounted to the sensor 20. FIG. 7A is a plan view of the sensor 20, and FIG. 7B is a cross-sectional view along line C-C in FIG. 7A. FIG. 8A is a plan view of the sensor 20 and the cable holder 30, and FIG. 8B is a cross-sectional view along line D-D in FIG. 8A. FIG. 9A is a cross-sectional view along line H-H in FIG. 9B, and FIG. 9B is a side view of the sensor 20 and the cable holder 30.

As illustrated in FIG. 4A, the sensor 20 has a structure in which four terminals 22 are provided on the terminal array surface 21a of the sensor body 21. Examples of the sensor 20 include a rectangular, thin plate-shaped chip-type CMOS image sensor, a pressure sensor, and a temperature sensor with a thickness not greater than 1 mm. The periphery of each terminal 22 is covered by resist 25, and a solder ball 23 is provided on a circular surface not covered by the resist 25. The resist 25 serves a function of protecting the circuit such as preventing short circuiting by a solder bridge when applied to a portion that is not soldered, and is used directly as an insulating film without peeling after soldering.

As illustrated in FIG. 4B, a UV curing adhesive 24 is applied to the center portion of the terminal array surface 21a of the sensor body 21 at a position that does not overlap with the solder balls 23 or the terminals 22. The UV curing adhesive 24 is applied at a lower height than the solder balls 23, which will be described below.

As illustrated in FIGS. 5 and 6, the cable holder 30 is fixed to such a sensor 20. That is, the cable holder 30 is mounted on the terminal array surface 21a of the sensor body 21 so that the connection grooves 32 surround the solder balls 23 of the sensor 20. At this time, the solder balls 23 are housed in the connection grooves 32 from the opening of the connection surface 31a.

As illustrated in FIG. 5, the UV curing adhesive 24 of the sensor 20 is applied so as to correspond to the central portion of the connection surface 31a of the cable holder 30. The connection surface 31a of the cable holder 30 of the first embodiment is formed with a flat profile, and when the cable holder 30 is mounted on the terminal array surface 21a of the sensor body 21, as illustrated in FIG. 6, the UV curing adhesive 24 spreads roughly to the connection surface 31a of the cable holder 30, excluding the regions of the solder balls 23. The cable holder 30 and the sensor 20 are adhered and fixed by irradiating the UV curing adhesive 24 extending between the terminal array surface 21a and the connection surface 31a with UV light.

The fixing of the sensor 20 and the cable holder 30 will be described further with reference to FIGS. 7A to 9B. First, the cross-sectional structure of the sensor will be described again using FIGS. 7A and 7B. The periphery of each terminal 22 provided on the terminal array surface 21a of the sensor body 21 is covered by a resist 25. At the boundary between the resist 25 and the terminal 22, the resist 25 is formed in an embankment shape protruding to cover the outer edge of the terminal 22. A solder ball 23 is provided on a circular surface of the terminal 22 not covered by the resist 25.

The UV curing adhesive 24 is applied to the central portion of the terminal array surface 21a of the sensor body 21, however, as illustrated in FIG. 7B, the height thereof is also set to be lower than that of the solder balls 23. Conversely, the solder balls 23 are set so that the height thereof is greater than that of the UV curing adhesive 24. As a result, when the cable holder 30 is mounted on the terminal array surface 21a of the sensor 20, the connection surface 31a is housed in the connection grooves 32 before coming into contact with the UV curing adhesive 24, and the UV curing adhesive 24 does not rise above the solder balls 23, whereby the upper surface of the solder balls can be prevented from being covered by the UV curing adhesive 24.

Next, the cross-sectional structure after the cable holder 30 is mounted on the terminal array surface 21a of the sensor 20 will be described using FIGS. 8A, 8B, 9A, and 9B. The width (diameter) L1 of the connection grooves 32 of the cable holder 30 is set to be greater than the width (diameter) L2 of the solder balls 23 of the sensor 20 so that when the cable holder 30 is mounted on the terminal array surface 21a, the solder balls 23 can be smoothly housed in the connection grooves 32.

Such a solder ball 23 prevents the terminal 22 from being covered by the UV curing adhesive 24 as a conductive body formed with a convex shape from the terminal 22, and ensures the electrical connection between the core wire 42 and the terminal 22.

When such a convex conductive body is formed from a solder ball 23, the solder ball 23 melts and becomes integral with the solder of the connection groove 32 after soldering, however, the tip of the core wire 42 is connected by soldering while separated from the upper surface of the terminal 22 by the height of the solder ball 23, and solder can be added to the connection groove 32 when the amount of solder of the solder ball 23 is insufficient.

The UV curing adhesive that is pushed out between the terminal array surface 21a and the connection surface 31a forms an adhesive layer for fixing the sensor 20 and the cable holder 30.

As illustrated in FIG. 8A, the area of the connection surface 31a and the cable extension surface 31b of the cable holder 30 is set to be smaller than the area of the terminal array surface 21a of the sensor 20, and the cable holder 30 is thus sized so as to not protrude from the sensor 20.

In the production process of the cable assembly 10, when the core wire 42 of the cable 40 is soldered to the plating of the connection groove 32, a pair of first side surfaces 31c of the parallel, flat cable holder 30 are pressed by a jig from the outside so that the core wire 42 does not protrude from the connection groove 32. The cable holder 30, however, is sized to be smaller than the sensor 20 and to not protrude from the sensor 20 so as to maximally use the space around the sensor 20.

In addition, in the production process of the cable assembly 10, when the cable holder 30 is mounted on the sensor 20, the connection surface 31a and the cable extension surface 31b of the cable holder 30 are formed in parallel and in a flat manner, so the cable holder 30 can be easily handled with a suction chuck jig such as a chip mounting machine, which makes it possible to improve productivity.

Since the UV curing adhesive 24 is applied to the terminal array surface 21a of the sensor 20 lower than the solder balls 23, when the cable holder 30 is mounted on the sensor 20, as illustrated in FIG. 8B in a cross-sectional view, the upper parts of the solder balls are housed in the connection grooves 32 and then pressed into the connection surface 31a of the cable holder 30 and pressed between the terminal array surface 21a and the connection surface 31a in a state lower than the solder balls 23 so as to be smoothened. Therefore, the UV curing adhesive 24 does not cover the upper surface of the solder balls, and does not hinder the soldering of the core wire 42 of the cable 40 to the solder balls 23 and the connection grooves 32. In addition, in a plan view, as illustrated in FIG. 8A, the UV curing adhesive 24 is the connection surface 31a of the cable holder 30 and spreads roughly in a range excluding the openings formed by the connection grooves 32, which makes it possible to reliably fix the cable holder 30 to the sensor 20. Note that FIG. 9A illustrates a cross section along line H-H passing through the solder balls 23 (see FIG. 9B) in FIG. 6.

Next, a cable assembly 10 of a second embodiment will be described with reference to FIGS. 10 to 12B. The second embodiment is the same as the first embodiment with the exception that the configuration of the cable holder 30 is different, so the second embodiment will be described with focus on the cable holder 30.

As illustrated in FIG. 10, the cable holder 30 of the second embodiment has an adhesive groove 33 in the connection surface 31a to allow the flow of the UV curing adhesive 24. Here, the connection groove 33 is a cross-shaped mode in which a protruding first adhesive groove 33a, which extends in a direction parallel to the first side surface 31c and opens toward the second side surface 31d, and a protruding second adhesive groove 33b, which extends in a direction parallel to the second side surface 31d and opens toward the first side surface, are orthogonal to one another. Note that the form of the adhesive groove 33 may be set appropriately out of consideration of the planar shape of the sensor 20 and the corresponding cable holder 30, or the positions and number of terminals 22.

When the cable holder 30 is mounted on the sensor 20, the UV curing adhesive 24 flows into the second adhesive groove 33b (also the same for the first adhesive groove 33a), as illustrated in FIG. 11B in a cross-sectional view. When viewed in a plan view, as illustrated in FIG. 12A, the UV curing adhesive 24 flows into the first adhesive groove 33a and the second adhesive groove 33b and spreads out in a cross shape. That is, the adhesive groove functions as a reservoir for the excess UV curing adhesive 24 so as to prevent the UV curing adhesive from protruding into unnecessary portions. In addition, the adhesive groove 33 makes it possible to induce the movement of the UV curing adhesive 24.

Further, since the contact area of the UV curing adhesive 24 and the cable holder 30 is increased by the adhesive groove 33, the adhesive force between the cable holder 30 and the sensor 20 increases.

Next, a cable assembly 10 of a third embodiment will be described with reference to FIGS. 13A to 14B. The third embodiment is the same as the first embodiment with the exception that the configuration of the sensor 20 is different, so the third embodiment will be described with focus on the sensor 20.

As illustrated in FIGS. 13A and 13B, the sensor 20 of the third embodiment has bumps 26 formed thereon in addition to the PAD or conductive pattern of the first embodiment serving as the terminals 22. The bumps 26 protrude from the terminal array surface 21a of the sensor body 21, and a copper or aluminum metal, for example, may be used as the material thereof. In addition, the bumps can be formed by continuous lamination from the terminals 22. That is, the bumps 26 are formed as convex conductive bodies similar to the solder balls 23.

The fixing of the sensor 20 and the cable holder 30 will be described using FIGS. 14A and 14B. As illustrated in FIG. 14B, the bumps 26 provided on the terminal array surface 21a of the sensor 20 are set to a height greater than that of the UV curing adhesive 24. As a result, when the cable holder 30 is mounted on the sensor 20, the UV curing adhesive 24 can be prevented from covering the upper surfaces of the bumps 26. The periphery of each bump 26 is covered by a resist 25.

The width (diameter) L1 of the connection grooves 32 of the cable holder 30 is set to be greater than the width (diameter) L3 of the bumps 26 of the sensor 20 so that when the cable holder 30 is mounted on the sensor 20, the bumps can be smoothly and easily housed in the connection grooves 32.

When the cable holder 30 is mounted on the sensor 20, as in the first embodiment, the upper parts of the bumps 26 are housed in the connection grooves 32 before the connection surface 31a of the cable holder 30 comes into contact with the UV curing adhesive 24. Therefore, in a cross-sectional view, as illustrated in FIG. 14B, the UV curing adhesive 24 is smoothened in a state lower than the bumps 26 so that it does not inhibit the soldering of the core wire 42 of the cable 40 to the bumps 26 and the connection grooves 32. In addition, in a plan view, as illustrated in FIG. 14A, the UV curing adhesive 24 spreads roughly to the connection surface 31a of the cable holder, with the exception of the regions of the bumps 26, so that the cable holder 30 is reliably adhered to the sensor 20. In this case, the bumps 26 are housed in the connection grooves 32 as convex conductive bodies.

Note that in FIG. 14B, the connection surface 31a of the cable holder 30 is illustrated as a flat surface, as in the first embodiment, however, a cable holder 30 having an adhesive groove 33 as in the second embodiment can also be combined with the sensor 20 of the third embodiment.

First, the method for fixing the cable holder 30 to the sensor is as follows. As a first step, a plurality of terminal array surfaces 21a of the sensor 20 are arranged in an upward orientation, and the UV curing adhesive 24 is applied lower than the solder balls 23 or bumps 26 so as to mount the cable holder 30. As a second step, the cable holder 30 is irradiated with UV from the upper surface on the cable holder 30 side so as to adhere and fix the sensor 20 and the cable holder 30. In this process, operations can be performed with high precision and in large volumes using a suction chuck jig of a chip mounting machine.

Next, as a third step, the sensor 20, where the cable holder 30 is fixed, is fixed to the jig. As a fourth step, the cable sheath 41 of the cable 40 is stripped to expose the core wire 42. As a fifth step, the core wire 42 of the cable 40 is inserted into the connection groove 32 of the cable holder 30 and is sandwiched by a flat plate-shaped jig so that the core wire 42 does not deviate from the connection groove 32 from both outer sides of the first side surface 31c. As a sixth step, solder is applied to the connection groove 32 of the cable holder 30. As a seventh step, the solder is melted by reflow so as to electrically connect and fix the sensor 20, the cable holder 30, and the cable 40.

Preferred embodiments of the present disclosure have been described in detail, however, the present disclosure is not limited to the embodiments described above, and various modifications and changes are possible within the scope of the gist of the present disclosure described in the scope of the patent claims.

For example, in the embodiments described above, a mode in which four cables 40 are separately inserted into the connection grooves 32 of the cable holder 30 was described, however, a plurality of cables 40 may be bundled together and inserted into the connection grooves 32 simultaneously, for example. This makes it possible to simplify the connection of the cables 40.

In addition, although four cables 40 were described in the above embodiments, in FIGS. 4A to 6, for example, the sensor 20 may be expanded in the left-right direction to increase the number of terminals 22 in that direction, and the connection grooves 32 of the cable holder 30 may also be increased in number in the left-right direction accordingly. As a result, even if the number of cables 40 that need to be connected increases, this can be handled by applying the gist of the embodiments.

Further, although the shape of the sensor 20 was described as rectangular and the shape of cable holder 30 was described as having a substantially rectangular profile corresponding thereto, when a cylindrical sensor 20 is used, for example, the profile of the cable holder 30 may be curtailed to form an arc-shaped profile so as not to protrude from the sensor 20. This makes it possible to smoothly pass the sensor 20 and the cable holder 30 into the cylinder.

In addition, although the cable connection direction was described as a direction orthogonal to the terminal array surface 21a in the above embodiments, the direction is not necessarily orthogonal, and the direction of the connection grooves can be variously changed so that the cable can also be applied in a direction intersecting the terminal array surface 21a.

Further, although the adhesive was a UV curing adhesive and the irradiated light was UV in the embodiments described above, the present disclosure is not limited to this case, and visible light curing may also be used.

1. A cable assembly comprising:
an electronic device;
a cable connected to the electronic device; and
a cable holder for supporting the electronic device and the cable,
wherein the electronic device comprises a terminal array surface on which terminals to be electrically connected to the cable are arranged,
wherein the cable holder comprises a connection surface facing the terminal array surface, and a side surface having a connection groove formed with an opening corresponding to the array positions of the terminals on the connection surface so as to support the cable in a direction intersecting the terminal array surface, and the connection groove is subjected to surface treatment to allow an electrical connection with a core wire of the cable,
wherein an adhesive layer is provided between the terminal array surface and the connection surface, and
wherein the cable holder is translucent.

2. The cable assembly according to claim 1, wherein a convex conductive body housed in the connection groove is provided on the terminal, and wherein the connection grove is set to have a greater width than the convex conductive body.

3. A cable holder comprising:
a connection surface which is a plane facing a terminal array surface on which terminals of an electronic device to which a cable is to be connected are arranged;
a cable extension surface from which the signal cable extends; and
a side surface connecting the connection surface and each side of the cable extension surface,
wherein the side surface includes a connection groove formed continuously so as to open toward the side surface side by forming an opening in the connection surface and the cable extension surface, and wherein the holder is translucent.

4. The cable holder according to claim 3, wherein the cable holder has an adhesive groove on the connection surface of the cable holder facing the terminal array surface of the electronic device.

5. A production method for a cable assembly comprising:
arranging a plurality of terminal array surfaces on which terminals of an electronic device are arranged so as to face upward;
applying a photocurable adhesive to locations other than the terminals of the terminal array surface lower than convex conductive bodies formed on the terminals;
mounting a translucent cable holder so that the applied photocurable adhesive spreads to the terminal array surfaces;
adhesively fixing the electronic device and the cable holder by irradiating light from the cable holder side; installing a core wire of the cable in a connection groove formed in the cable holder; and
connecting the core wire, the connection groove, and the terminals.

6. The production method for a cable assembly according to claim 5, wherein an upper portion of the convex conductive body is housed in the connection groove of the cable holder, and the cable holder then comes into contact with the photocurable adhesive.
